(19) United States
(12) Patent Application Publication
(10) Pub. No.: US 2017/0260501 A1
(43) Pub. Date: Sep. 14, 2017

(54) DERIVATION OF NEURAL CREST STEM CELLS AND USES THEREOF

(71) Applicant: International Stem Cell Corporation, Carlsbad, CA (US)

(72) Inventors: Ruslan Semechkin, Carlsbad, CA (US); Rodolfo Gonzalez, Carlsbad, CA (US)

(21) Appl. No.: 15/529,490

(22) PCT Filed: Nov. 24, 2015
(86) PCT No.: PCT/US15/62519

§ 371 Date: May 24, 2017

Related U.S. Application Data
(60) Provisional application No. 62/084,286, filed on Nov. 25, 2014.

(51) Int. Cl. C12N 5/0735; G01N 33/50; C12N 5/07; C12N 5/0775

(52) U.S. Cl. CPC C12N 5/0606; C12N 5/0662; G01N 33/5058; C12N 5/06; C12N 2506/02

(57) ABSTRACT

The present invention is based in part the discovery of methods for the generation of neural crest stem cells (NCSCs) from human pluripotent stem cells (hPSCs). Specifically, the present invention discloses methods for the use of a combination of rho-associated protein kinase (ROCK) inhibitors, glycogen synthase kinase 3 (GSK-3) inhibitors, activing receptor like kinase (ALK) receptor inhibitors and bone morphogenic protein (BMP) receptor inhibitors to derive NCSCs from hPSCs. The present invention also discloses methods to treat neurocristopathic diseases and disorders using NCSCs derived from hPSCs.

### CROSS REFERENCE TO RELATED APPLICATIONS

This application claims the benefit of priority under §119(e) to U.S. Ser. No. 62/084,286, filed Nov. 25, 2014. The disclosure of the prior application is considered part of and is incorporated by reference in its entirety in the disclosure of this application.

### FIELD OF THE INVENTION

The invention relates generally to stem cells and more specifically to the derivation of neural crest stem cells (NCSCs) from human pluripotent stem cells (hPSCs) and the use of the NCSCs for the treatment of neurocristopathic disorders or diseases.

### BACKGROUND INFORMATION

Human pluripotent stem cells (hPSCs) are cells that can differentiate into a large array of cell types. Stem cells are distinguished from other cell types by two important characteristics. First, they are unspecialized cells capable of renewing themselves through cell division, sometimes after long periods of inactivity. Second, under certain physiologic or experimental conditions, they can be induced to become tissue- or organ-specific cells with special functions. In some organs, such as the gut and bone marrow, stem cells regularly divide to repair and replace worn out or damaged tissues. In other organs, however, such as the pancreas and the heart, stem cells only divide under special conditions.

During embryonic development, stem cells form the tissues of the body from three major cell populations: ectoderm, mesoderm and definitive endoderm. Mesoderm gives rise to blood cells, endothelial cells, cardiac and skeletal muscle, and adipocytes. Definitive endoderm generates liver, pancreas and lung. Ectoderm gives rise to the nervous system, skin and adrenal tissues.

Stem cells have potential in many different areas of health and medical research. Some of the most serious medical conditions, such as cancer and birth defects, are due to problems that occur when cells undergo a transformation. Understanding normal cell development and differentiation mechanisms will allow for a better understanding of these conditions.

Neurocristopathy is a diverse class of pathologies that arise from defects in the development of tissues containing cells commonly derived from the embryonic neural crest cell lineage. Common neurodegenerative diseases include Waardenburg syndrome and Hirschsprung disease. For example, Waardenburg syndrome is a rare genetic disorder most often characterized by varying degrees of deafness, minor defects in structures arising from the neural crest, and pigmentation anomaly. There is currently no cure for Waardenburg syndrome and other abnormalities associated with the syndrome are treated symptomatically. Because Waardenburg syndrome arises from defects in the neural crest, there are opportunities for cell replacement therapy by implanting neural crest stem cells or cells derived from neural crest stem cells into Waardenburg syndrome patients.

The generation of neural crest stem cells (NCSCs) from human pluripotent stem cells (hPSC), such as hESC, hiPSC or parthenogenetic stem cells (hpSC), is a vital component of cell-based strategies for the treatment of Neurocristopathies. However, before hPSC-derived NCSCs can be administered in therapeutic modalities, chemically defined culture conditions must be developed that reproducibly and robustly induce the generation of NCSCs.

### SUMMARY OF THE INVENTION

The present invention is based in part the discovery of methods for the generation of neural crest stem cells (NCSCs) from human pluripotent stem cells (hPSCs). Specifically, the present invention discloses methods for the use of a combination of a rho-associated protein kinase inhibitor, a glycogen synthase kinase 3 (GSK-3) inhibitor, an activing receptor-like kinase (ALK) receptor inhibitor and a bone morphogenic protein (BMP) receptor inhibitor to derive NCSCs from hPSCs. The present invention also discloses methods to treat neurocristopathic diseases and disorders using NCSCs derived from hPSCs.

In one embodiment, the present invention provides for a method of differentiating human pluripotent stem cells (hPSCs) into neural crest stem cells (NCSCs) comprising culturing hPSCs with at least two agents including a rho-associated protein kinase (ROCK) inhibitor, a glycogen synthase kinase 3 (GSK-3) inhibitor, an activing receptor-like kinase (ALK) receptor inhibitor and/or a bone morphogenic protein (BMP) receptor inhibitor, under conditions for such time as to allow the agents to effect differentiation of the hPSCs. In one aspect, the hPSCs are parthenogenetic stern cells (hpSCs), induced pluripotent stem cells (iPSCs), nuclear transfer stem cells, adult stem cells or embryonic stem cells. In another aspect, the ALK inhibitor inhibits ALK4, ALK5 and/or ALK7 and the BMP receptor inhibitor inhibits ALK2. In an additional aspect, the ROCK inhibitor is Y27632, AS1 892802, GSK 269962, GSK 429286, H 1152, HA 1100 hydrochloride, OXA 06 dihydrochloride, RKI 1447 dihydrocholoride, SB 772077B dihydrocholoride, SR 3677 dihdrochloride, or TC-S 7001, the GSK-3 inhibitor is Chir99021, 3F8, A 1070722, AR-A 014418, BIO, BIO-acetoxime, 10Z-Hymenialdisine, Indirubin-3′-oxime, Kenpaullone, Lithium carbonate, NSC 693868, SB216763, SB 415286, TC-G 24, TCS 2002, TCS21311, or TWS 119, the ALK inhibitor is SB43152, A 83-01, D 4476, GW 788388, LY 364974, R 268712, RepSox, SB 505124, SB 525334, or SD 208 and the BMP receptor inhibitor is DMH-1, DMH2, Dorsomorphin dihydrochloride, K 02288, or ML 347. In a further aspect, the hPSCs are contacted with at least three agents. In a specific aspect, the at least three agents are Y27632, Chir99021, SB43152 and/or DMH-1. In an additional aspect, the hPSCs are contacted with at least four agents. In a specific aspect, the at least four agents are Y27632, Chir99021, 51343152 and DMH-1. In a further aspect, the NCSCs express at least one neural crest cell marker and at least one marker of pluripotency. In one aspect, the at least one neural crest cell marker of differentiation is PAX3, P75, NGFR, SOX10, FOXD3, NESTIN, SNAI2, Ki67 or HNK-1 and the at least one marker of pluripotency is NANOG, ZNF206, or OCT4. In an additional aspect, the hPSCs are contacted with the at least two agents for at least about 1, 2, 3, 4, 5, 6, 7, 8, 9, or 10 days. In a preferred aspect, the contacting is for at least about 6 days. In another aspect, the NCSCs are capable of being maintained in an undifferentiated state for at least about 1, 2, 3, 4, 5, 6, 7, 8, 9, 10, 15, 20 or 25 passages. In a specific aspect, the NCSCs are capable of being maintained in an undifferentiated state for at least about 5 passages. In an additional aspect, the NCSCs are differentiated into astrocytes, smooth muscle cells, osteoblast, adipocytes, chondrocytes, melanocytes, Schwann cells and/or neurons. In certain aspects, the astrocytes express S1001β, HNK1 and/or GFAP; the smooth muscle cells express Caldesmon, P75 and/or SMA and the neurons express MAP2, SOX10 and/or TUJ1. In one aspect, the hPSCs are cultured in a media comprising StemLife MSC basal medium, Glutamax, B27, Y27632, CHIR99021, SB43152 and DMH-1.

In another embodiment, the present invention provides a method of treating neurocristopathic disease or disorder comprising obtaining human pluripotent stem cells (hPSCs); contacting the hPSCs with at least two agents selected from the group consisting of a rho-associated protein kinase (ROCK) inhibitor, a glycogen synthase kinase 3 (GSK-3) inhibitor, an activing receptor-like kinase (ALK) receptor inhibitor and/or a bone morphogenic protein (BMP) receptor inhibitor to differentiate the hPSCs into neural crest stem cells (NCSCs) under conditions and for such time as to allow the agents to effect differentiation of the hPSCs; and administering the NCSCs to a subject in need thereof. In one aspect, the neurocristopathic disease or disorder is piebaldism, Waardenburg syndrome, Hirschsprung disease, Ondine's curse (congenital central hypoventilation syndrome), pheochromocytoma, paraganglioma, Merkel cell carcinoma, multiple endocrine neoplasia, neurofibromatosis type I, CHARGE syndrome, familial dysautonomia, DiGeorge syndrome, Axenfeld-Rieger syndrome, Goldenhar syndrome (a.k.a. hemifacial microsomia), craniofrontonasal syndrome, congenital melanocytic nevus, melanoma, or congenital heart defects of the outflow track. In another aspect, the neurocristopathic disease or disorder is Waardenburg syndrome or Hirschsprung disease. In an additional aspect, the hPSCs are parthenogenetic stem cells (hpSCs), induced pluripotent stem cells (iPSCs), nuclear transfer stem cells, adult stem cells or embryonic stern cells. In a further aspect, the ROCK inhibitor is Y27632, the GSK-3 inhibitor is Chir99021, ALK receptor inhibitor is SB43152 and the BMP receptor inhibitor is DMH-1. In one aspect, the NCSCs express at least one neural crest cell marker wherein the neural crest stem cell marker is PAX3, P75 NGFR, SOX10, FOXD3, NESTIN, SNAI2, Ki67 or HNK-1 and the at least one marker of pluripotency is NANOG, ZNF206, or OCT4. In another aspect, the contacting is for at least about 6 days and the NCSCs are capable of being maintained in an undifferentiated state for at least about 5 passages. In a further aspect, the NCSCs are differentiated into astrocytes, smooth muscle cells, osteoblast, adipocytes, chondrocytes, melanocytes, Schwann cells and/or neurons.

In an additional embodiment, the invention provides for a kit for the differentiation of human pluripotent stem cells (hPSCs) into neural crest stem cells (NCSCs) comprising of a rho-associated protein kinase (ROCK) inhibitor, a glycogen synthase kinase 3 (GSK-3) inhibitor, an activing receptor-like kinase (ALK) receptor inhibitor and a bone morphogenic protein (BMP) receptor inhibitor and instructions. In one aspect, ROCK inhibitor is Y27632, the GSK-3 inhibitor is Chir99021, the ALK receptor inhibitor is SB43152 and the BMP receptor inhibitor is DMH-1.

### BRIEF DESCRIPTION OF THE DRAWINGS

FIG. 1 is a schematic illustration that describes the derivation and proliferation of neural crest stem cells from parthenogenetic stem cells.

FIGS. 2A-2G are graphs of gene expression. The results show that human parthenogenetic derived neural crest stern cells (hpNCSCs) express genes associated with the neural crest lineage and markers of pluripotency. A. NANOG, B. OCT4, C. PAX3, D. P75 NGFR, E. SOX10, F. NESTIN, and G. SNAI2.

### DETAILED DESCRIPTION OF THE INVENTION

The present invention is based in part the discovery of methods for the generation of neural crest stem cells (NCSCs) from human pluripotent stem cells (hPSCs). Specifically, the present invention discloses methods for the use of a combination of a rho-associated protein kinase inhibitor, a glycogen synthase kinase 3 (GSK-3) inhibitor, an activing receptor-like kinase (ALK) receptor inhibitor and a bone morphogenic protein (BMP) receptor inhibitor to derive NCSCs from hPSCs. The present invention also discloses methods to treat neurocristopathic diseases and disorders using NCSCs derived from hPSCs.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention provides methods for the generation of neural crest stem cells (NCSCs) from human pluripotent stem cells (hPSCs) and the use of the derived NCSCs for the treatment of neurocristopathic disease or disorders.

Neural crest cells are a temporary group of cells unique to vertebrates that arise from the embryonic ectoderm cell layer, and in turn give rise to a diverse cell lineage including melanocytes, craniofacial cartilage and bone, smooth muscle, peripheral and enteric neurons and glia. The neural crest cells migrate extensively to generate a prodigious number of differentiated cell types. These cell types include (1) the neurons and glial cells of the sensory, sympathetic, and parasympathetic nervous systems, (2) the epinephrine-producing (medulla) cells of the adrenal gland, (3) the pigment-containing cells of the epidermis, and (4) many of the skeletal and connective tissue components of the head. The fate of the neural crest cells depends, to a large degree, on where they migrate to and settle. Neural crest cells give rise to cells including neurons, including sensory ganglia, sympathetic and parasympathetic ganglia, and plexuses neuroglial cells Schwann cells; adrenal medulla; calcitonin-secreting cells; epidermal pigment cells; facial and anterior ventral skull cartilage and bones; corneal endothelium and stroma; tooth papillae; dermis, smooth muscle, and adipose tissue of skin of head and neck; connective tissue of salivary, lachrymal, thymus, thyroid, and pituitary glands; and connective tissue and smooth muscle in arteries of aortic arch origin.

Neurocristopathy is a diverse class of pathologies that may arise from defects in the development of tissues containing cells commonly derived from the embryonic neural crest cell lineage. Examples of neurocristopathic diseases and disorders include piebaldism, Waardenburg syndrome, Hirschsprung disease, Ondine's curse (congenital central hypoventilation syndrome), pheochromocytoma, paraganglioma, Merkel cell carcinoma, multiple endocrine neoplasia, neurofibromatosis type I, CHARGE syndrome, familial dysautonomia, DiGeorge syndrome, Axenfeld-Rieger syndrome, Goldenhar syndrome (a.k.a. hemifacial microsomia), craniofrontonasal syndrome, congenital melanocytic nevus, melanoma, and certain congenital heart defects of the outflow tract, in particular. Additionally, multiple sclerosis has also been suggested as being neurocristopathic in origin.

The generation of neural crest stem cells (NCSCs) from human pluripotent stem cells (hPSC), such as hESC, hiPSC or parthenogenetic stem cells (hpSC), is a vital component of cell-based strategies for the treatment of Neurocristopathies. However, before hPSC-derived NCSCs can be administered in therapeutic modalities, chemically defined culture conditions must be developed that reproducibly and robustly induce the generation of NCSCs. The methods reported provides instructions for the generation of a homogenous population of NCSCs from hPSCs that can be expanded, frozen and further differentiated into bone, muscle, cartilage, nerves, endothelium and connective tissue for cell therapies or drug discovery.

As used herein, “neurocristopathic disease or disorder” refers to any disease or disorder which is characterized by defects arising from neural crest cells. Examples of neurocristopathic diseases or disorders include, but are not limited to, is piebaldism, Waardenburg syndrome, Hirschsprung disease, Ondine's curse (congenital central hypoventilation syndrome), pheochromocytoma, paraganglioma, Merkel cell carcinoma, multiple endocrine neoplasia, neurofibromatosis type I, CHARGE syndrome, familial dysautonomia, DiGeorge syndrome, Axenfeld-Rieger syndrome, Goldenhar syndrome (a.k.a. hemifacial microsomia), craniofrontonasal syndrome, congenital melanocytic nevus, melanoma, and congenital heart defects of the outflow track.

Waardenburg syndrome is a rare genetic disorder most often characterized by varying degrees of deafness, minor defects in structures arising from the neural crest, and pigmentation anomalies. Mutations in the EDN3, EDNRB, MITF, PAX3, SNAI2, and SOX10 genes are implicated in Waardenburg Syndrome. Some of these genes are involved in the making of melanocytes, which makes the pigment melanin. Melanin is an important pigment in the development of hair, eye color, skin, and functions of the inner ear. So the mutation of these genes can lead to abnormal pigmentation and hearing loss. PAX3 and MTIF gene mutation occurs in type I and II (WS1 and WS2). Type III (WS3) shows mutations of the PAX3 gene also. SOX10, EDN3, or EDNRB gene mutations occur in type IV. Type IV (WS4) can also affect portions of nerve cell development that potentially can lead to intestinal issues. Waardenburg syndrome is characterized by sensorineural hearing loss, iris pigmentary abnormality (heterochromia iridum—different colors of iris in two eyes or heterochromia iridis—two different colors of iris in same eye or characteristic brilliant blue iris), hair hypopigmentation (white forelock or white hairs at other sites on the body) (poliosis), and dystopia canthorum (lateral displacement of inner canthi). There is currently no treatment or cure for Waardenburg syndrome. The symptom most likely to be of practical importance is deafness, and this is treated as any other irreversible deafness would be. In marked cases there may be cosmetic issues. Other abnormalities (neurological, structural, Hirschsprung disease) associated with the syndrome are treated symptomatically.

Hirschsprung disease is a form of megacolon that occurs when part or all of the large intestine or antecedent parts of the gastrointestinal tract have no ganglion cells and therefore cannot function. During normal prenatal development, cells from the neural crest migrate into the large intestine (colon) to form the networks of nerves called the myenteric plexus (Auerbach plexus) (between the smooth muscle layers of the gastrointestinal tract wall) and the submucosal plexus (Meissner plexus) (within the submucosa of the gastrointestinal tract wall). In Hirschsprung's disease, the migration is not complete and part of the colon lacks these nerve bodies that regulate the activity of the colon. The affected segment of the colon cannot relax and pass stool through the colon, creating an obstruction. In most affected people, the disorder affects the part of the colon that is nearest the anus. In rare cases, the lack of nerve bodies involves more of the colon. In five percent of cases, the entire colon is affected. Stomach and esophagus may be affected too. Treatment of Hirschsprung's disease consists of surgical removal (resection) of the abnormal section of the colon, followed by reanastomosis.

The methods of deriving neural crest stem cells (NCSCs) that are described herein are generated from human pluripotent stern cells (hPSCs), such as embryonic stem cells. As used herein, “embryonic” refers to a range of developmental stages of an organism beginning with a single zygote and ending with a multicellular structure that no longer comprises pluripotent or totipotent cells other than developed gametic cells. In addition to embryos derived by gamete fusion, the term “embryonic” refers to embryos derived by somatic cell nuclear transfer. Human stem cells can be maintained in culture in a pluripotent state without substantial differentiation using methods that are known in the art. Such methods are described, for example, in U.S. Pat. Nos. 5,453,357, 5,670,372, 5,690,926 5,843,780, 6,200,806 and 6,251,671 the disclosures of which are incorporated herein by reference in their entireties.

As used herein, “multipotent” or “multipotent cell” refers to a cell type that can give rise to a limited number of other particular cell types. Examples of multipotent cells include ectodermal cells, endodermal cells, mesodermal cells and neural stem cells which can give rise to limited number of other cells.

As used herein, a “pluripotent cell” refers to a cell that can be maintained in vitro for prolonged, theoretically indefinite period of time in an undifferentiated state, that can give rise to different differentiated tissue types, i.e., ectoderm, mesoderm, and endoderm. Human pluripotent stem cells (hPSCs) include, but are not limited to, human embryonic stem cells (hESCs), human parthenogenetic stem cells (hpSCs), nuclear transfer stem cells, adult stem cells and induced pluripotent stem cells (iPSCs). Methods of obtaining such hPSCs are well known in the art.

One method of obtaining hPSCs is by parthenogenesis. “Parthenogenesis” (“parthenogenically activated” and “parthenogenetically activated” is used herein interchangeably) refers to the process by which activation of the oocyte occurs in the absence of sperm penetration, and refers to the development of an early stage embryo comprising trophectoderm and inner cell mass that is obtained by activation of an oocyte or embryonic cell, e.g., blastomere, comprising DNA of all female origin. In a related aspect, a “parthenote” refers to the resulting cell obtained by such activation. In another related aspect, “blastocyst” refers to a cleavage stage of a fertilized of activated oocyte comprising a hollow ball of cells made of outer trophoblast cells and an inner cell mass (ICM). In a further related aspect, “blastocyst formation” refers to the process, after oocyte fertilization or activation, where the oocyte is subsequently cultured in media for a time to enable it to develop into a hollow ball of cells made of outer trophoblast cells and ICM (e.g., 5 to 6 days). One method for generating hpSCs comprises parthenogenetically activating a human oocyte by contacting the oocyte with an ionophore at high O₂ tension and contacting the oocyte with a serine-threonine kinase inhibitor under low O₂ tension; cultivating the activated oocyte at low O₂ tension until blastocyst formation; transferring the blastocyst to a layer of feeder cells, and culturing the transferred blastocyst under high O₂ tension; mechanically isolating an inner cell mass (ICM) from trophectoderm of the blastocyst and culturing the cells of the ICM on a layer of feeder cells under high O₂ tension, thereby producing human parthenogenetic stem cells.

Another method of obtaining hPSCs is through nuclear transfer. As used herein, “nuclear transfer” refers to the fusion or transplantation of a donor cell or DNA from a donor cell into a suitable recipient cell, typically an oocyte of the same or different species that is treated before, concomitant or after transplant or fusion to remove or inactivate its endogenous nuclear DNA. The donor cell used for nuclear transfer include embryonic and differentiated cells, e.g., somatic and germ cells. The donor cell may be in a proliferative cell cycle (G1, G2, S or M) or non-proliferating (G0 or quiescent). Preferably, the donor cell or DNA from the donor cell is derived from a proliferating mammalian cell culture, e.g., a fibroblast cell culture. The donor cell optionally may be transgenic, i.e., it may comprise one or more genetic addition, substitution or deletion modifications.

A further method for obtaining hPSCs is through the reprogramming of cells to obtain induced pluripotent stem cells (iPSCs). Takahashi et al. (Cell 131, 861-872 (2007)) have disclosed methods for reprogramming differentiated cells, without the use of any embryo or ES (embryonic stem) cell, and establishing an inducible pluripotent stem cell having similar pluripotency and growing abilities to those of an ES cell. Takahashi et al. describe various different nuclear reprogramming factors for differentiated fibroblasts, which include products of the following four genes: an Oct family gene; a Sox family gene; a Klf family gene; and a Myc family gene.

Adult stem cells are another source from hPSCs. Adult stem cells are undifferentiated cells, found throughout the body after development, that multiply by cell division to replenish dying cells and regenerate damaged tissues. Adult stem cells have the ability to divide or self-renew indefinitely, and generate all the cell types of the organ from which they originate, potentially regenerating the entire organ from a few cells. Adult stem cells include hematopoietic stem cells, mammary stem cells, intestinal stem cells, mesenchymal stem cells, endothelial stem cells, neural stem cells, olfactory adult stem cells, neural crest stem cells and testicular cells. Methods of isolating adult stem cells are well known in the art.

The pluripotent state of the cells is preferably maintained by culturing cells under appropriate conditions, for example, by culturing on a fibroblast feeder layer or another feeder layer or culture that includes leukemia inhibitory factor (LIF). The pluripotent state of such cultured cells can be confirmed by various methods, e.g., (i) confirming the expression of markers characteristic of pluripotent cells; (ii) production of chimeric animals that contain cells that express the genotype of the pluripotent cells; (iii) injection of cells into animals, e.g., SCID mice, with the production of different differentiated cell types in vivo; and (iv) observation of the differentiation of the cells (e.g., when cultured in the absence of feeder layer or LIF) into embryoid bodies and other differentiated cell types in vitro.

The pluripotent state of the cells used in the present invention can be confirmed by various methods. For example, the cells can be tested for the presence or absence of characteristic ES cell markers. In the case of human ES cells, examples of such markers are identified supra, and include NANOG, SSEA-4, SSEA-3, TRA-1-60, TRA-1-81, ZNF206, and OCT 4, and are known in the art.

The resultant pluripotent cells and cell lines, preferably human pluripotent cells and cell lines have numerous therapeutic applications. Such pluripotent cells may be used for cell transplantation therapies or gene therapy (if genetically modified) in the treatment of numerous disease conditions.

Human pluripotent stem cells (hPSCs) include, but are not limited to, human embryonic stem cells, human parthenogenetic stem cells, induced pluripotent stem cells, adult stem cells and nuclear transfer cells and cell lines produced by such cells. hPSCs are maintained in culture in a pluripotent state by routine passage until it is desired that neural crest stem cells be derived. Examples of human parthenogenetic stem cell lines include LLC2P and LLC12PH.

An “NCSC” (also referred to as a “multipotent neural crest stem cell”) exhibits one or more of the following properties: 1) the expression of Nestin, SOX10, HNK-1, P75, PAX3, SNAI2, and/or Ki67; 2) the ability to undergo self-renewal; 3) ability to differentiate into cell types such as astrocytes, smooth muscle and neurons; and 6) morphological characteristics typical for NCSCs.

NCSCs are self-renewing, multipotent cells that generate a wide variety of cell types. Under the appropriate conditions, NCSCs can differentiate into astrocytes, smooth muscle and neurons.

As used herein, “agent” is a compound that induces an hPSCs to become a NCSC. Agents may be small molecules or other chemical compounds. Such small molecules include, but are not limited to, rho-associated protein kinase (ROCK) inhibitors, glycogen synthase kinase 3 (GSK-3) inhibitors, activing receptor-like kinase (ALK) receptor inhibitors and bone morphogenic protein (BMP) receptor inhibitors.

Rho-associated protein kinase (ROCK) is a kinase belonging to the AGC (PKA/PKG/PKC) family of serine-threonine kinases. ROCK plays a role in a wide range of different cellular phenomena, as ROCK is a downstream effector protein of the small GTPase Rho, which is one of the major regulators of the cytoskeleton. Examples of ROCK inhibitors include, but are not limited to, AS 1892802, GSK 269962, GSK 429286, H 1152, HA 1100, OXA 06, RKI 1447, SB 772077B, SR3677, TC-S7001 and Y27632.

Glycogen synthase kinase 3 is a serine/threonine protein kinase that mediates the addition of phosphate molecules onto serine and threonine amino acid residues. GSK-3 functions by phosphorylating a serine or threonine residue on its target substrate. A positively charged pocket adjacent to the active site binds a “priming” phosphate group attached to a serine or threonine four residues C-terminal of the target phosphorylation site. The active site, at residues 181, 200, 97, and 85, binds the terminal phosphate of ATP and transfers it to the target location on the substrate. Phosphorylation of a protein by GSK-3 usually inhibits the activity of its downstream target. GSK-3 is active in a number of central intracellular signaling pathways, including cellular proliferation, migration, glucose regulation, and apoptosis. Examples of GSK-3 inhibitors include, but are not limited to, A 1070722, AR-A 014418, CHIR 99021, BIO, BIO-acetoxime, Kenpaullone, TWS 119, AR-A 014418, SB 415286, TCS 21311, Lithium carbonate, 3F8, L803, Indirubin-3′-oxime, 10Z-Hymenialdisine, L803-mts, NSC 693868, SB216763, TC-G 24, TCS 2002, and TWS 119.

Transforming growth factor-beta (TGFbeta) regulates the activation state of the endothelium via two opposing type I receptor/Smad pathways. Activin receptor-like kinase-1 (ALK1) induces Smad1/5 phosphorylation, leading to an increase in endothelial cell proliferation and migration, while ALK5 promotes Smad2/3 activation and inhibits both processes. Examples of ALK receptors inhibitors include, but are not limited to, A 83-01, Crizotinib, TAE684, Alectinib, Ceritinib, AP26113, GSK1838705A, AZD3463, ASP3026, SB43152, D 4476, GW 788388, LY 364974, R 268712, RepSox, SB 505124, SB 525334, and SD 208.

Bone morphogenetic proteins (BMPs) are a group of growth factors also known as cytokines and as metabologens. BMPs interact with specific receptors on the cell surface, referred to as bone morphogenetic protein receptors (BMPRs). Signal transduction through BMPRs results in mobilization of members of the SMAD family of proteins. The signaling pathways involving BMPs, BMPRs and Smads are important in the development of the heart, central nervous system, and cartilage, as well as post-natal bone development. They have an important role during embryonic development on the embryonic patterning and early skeletal formation. As such, disruption of BMP signaling can affect the body plan of the developing embryo Examples of BMP inhibitors include, but are not limited to, DMH2, Dorsomorphin, LD-193189, DMH-1, K 02288, and ML 347.

Once NCSCs are derived, the cells may be maintained in vitro for prolonged, theoretically indefinite periods of time retaining the ability to differentiate into cell types, such as astrocytes, smooth muscle and neurons. As described in the Examples, hPSCs derived NCSCs can be passaged for at least 5 passaged in an undifferentiated state and differentiated into astrocytes, smooth muscle and neurons.

As used herein, “differentiation” refers to a change that occurs in cells to cause those cells to assume certain specialized functions and to lose the ability to change into certain other specialized functional units. Cells capable of differentiation may be any of totipotent, pluripotent or multipotent cells. Differentiation may be partial or complete with respect to mature adult cells.

“Differentiated cell” refers to a non-embryonic cell that possesses a particular differentiated, i.e., non-embryonic, state. The three earliest differentiated cell types are endoderm, mesoderm, and ectoderm.

NCSCs derived from PSCs are multipotent and can be differentiated into several cell types including astrocytes, smooth muscle, osteoblast, adipocytes, chondrocytes, melanocytes, Schwann cells and neurons.

A neuron is an electrically excitable cell that processes and transmits information through electrical and chemical signals. A chemical signal occurs via a synapse, a specialized connection with other cells. Neurons connect to each other to form neural networks. Neurons are the core components of the nervous system, which includes the brain, spinal cord, and peripheral ganglia. A number of specialized types of neurons exist: sensory neurons respond to touch, sound, light and numerous other stimuli affecting cells of the sensory organs that then send signals to the spinal cord and brain. Motor neurons receive signals from the brain and spinal cord, cause muscle contractions, and affect glands. Interneurons connect neurons to other neurons within the same region of the brain or spinal cord.

Neurons may be identified by expression of neuronal markers Tuj1 (beta-III-tubulin); MAP-2 (microtubule associated protein 2, other MAP genes such as MAP-1 or -5 may also be used); anti-axonal growth clones; ChAT (choline acetyltransferase); CgA (anti-chromagranin A); DARRP (dopamine and cAMP-regulated phosphoprotein); DAT (dopamine transporter); GAD (glutamic acid decarboxylase); GAP (growth associated protein); anti-HuC protein; anti-HuD protein; α-internexin; NeuN (neuron-specific nuclear protein); NF (neurofilament); NGF (nerve growth factor); γ-SE (neuron specific enolase); peripherin; PH8; PGP (protein gene product); SERT (serotonin transporter); synapsin; Tau (neurofibrillary tangle protein); anti-Thy-1; TRK (tyrosine kinase receptor); TRH (tryptophan hydroxylase); anti-TUC protein; TH (tyrosine hydroxylase); VRL (vanilloid receptor like protein); VGAT (vesicular GABA transporter), and/or VGLUT (vesicular glutamate transporter).

Astrocytes are characteristic star-shaped glial cells in the brain and spinal cord. The proportion of astrocytes in the brain is not well defined. Depending on the counting technique used, studies have found that the astrocyte proportion varies by region and ranges from 20% to 40% of all glia. They perform many functions, including biochemical support of endothelial cells that form the blood-brain barrier, provision of nutrients to the nervous tissue, maintenance of extracellular ion balance, and a role in the repair and scarring process of the brain and spinal cord following traumatic injuries.

Astrocytes may be identified by expression of specific markers including GFAP, S100β, S100A1, EAAT1, EAAT2, ALDH1L1, SR101, Survivin, ABCA1, ABCA7, NCAM-1, FKBP38, and/or KAT3B.

Smooth muscle is an involuntary non-striated muscle. It is divided into two subgroups; the single-unit (unitary) and multiunit smooth muscle. Within single-unit cells, the whole bundle or sheet contracts as a syncytium (i.e. a multinucleate mass of cytoplasm that is not separated into cells). Multiunit smooth muscle tissues innervate individual cells; as such, they allow for fine control and gradual responses, much like motor unit recruitment in skeletal muscle. Smooth muscle is found within the walls of blood vessels (such smooth muscle specifically being termed vascular smooth muscle) such as in the tunica media layer of large (aorta) and small arteries, arterioles and veins. Smooth muscle is also found in lymphatic vessels, the urinary bladder, uterus (termed uterine smooth muscle), male and female reproductive tracts, gastrointestinal tract, respiratory tract, arrector pili of skin, the ciliary muscle, and iris of the eye. The structure and function is basically the same in smooth muscle cells in different organs, but the inducing stimuli differ substantially, in order to perform individual effects in the body at individual times. In addition, the glomeruli of the kidneys contain smooth muscle-like cells called mesangial cells.

Smooth muscle cells may be identified by expression of specific markers including troponin, TPM3, tropomyosin, TMP2, TrkA, TrkB, Calponin, alpha-smooth muscle actin, VE-cadherin, caldesmon/CALD1, hexim1, histamine H2 R, MotilinR/GPR38 and/or transregulin/TAGLN.

Osteoblast are cells with single nuclei that synthesize bone. However, in the process of bone formation, osteoblasts function in groups of connected cells. Individual cells cannot make bone, and the group of organized osteoblasts together with the bone made by a unit of cells is usually called the osteon. Osteoblasts are specialized, terminally differentiated products of mesenchymal stem cells. The cells synthesize very dense, crosslinked collagen, and several additional specialized proteins in much smaller quantities, including osteocalcin and osteopontin, which compose the organic matrix of bone. In organized groups of connected cells, osteoblasts produce a calcium and phosphate-based mineral that is deposited, in a highly regulated manner, into the organic matrix forming a very strong and dense mineralized tissue—the mineralized matrix. The mineralized skeleton is the main support for the bodies of air breathing vertebrates and is also an important store of minerals for physiological homeostasis including both acid-base balance and calcium or phosphate maintenance.

Osteoblast cells may be identified by expression of specific markers including 5′-Nucleotidase/CD73, Aggrecan, ALCAM/CD166, Alkaline Phosphatase/ALPL, B220/CD45R, Biglycan, Calcitonin R, CD44, CD45, CD45.1, CD45.2, CD90/Thy1, CD45RO, Collagen I, DC-STAMP, Decorin, DLX5, DMP-1, EBF-2, Fibronectin, Fibronectin/Anastellin, GABA-B R1, IBSP/Sialoprotein II, IGFBP-3, IGFBP-rP10, Integrin alpha V/CD51, MEPE/OF45, NFIL3/E4BP4, OC-STAMP, OSCAR, Osteoadherin/OSAD, Osteocalcin, Osterix/Sp7, PTH1R/PTHR1, RANK/TNFRSF11A, RUNX2/CBFA1, SCUBE3, SMOC-1, SMOC-2, SPARC, TBX2, TBX3, TBX5, TCIRG1, Thrombopoietin/Tpo, TRACP/PAP/ACP5, TRANCE/TNFSF11/RANK L, USAG1 and/or WDR5.

Adipocytes, also known as lipocytes and fat cells, are the cells that primarily compose adipose tissue, specialized in storing energy as fat. There are two types of adipose tissue, white adipose tissue (WAT) and brown adipose tissue (BAT), which are also known as white fat and brown fat, respectively, and comprise two types of fat cells. Most recently, the presence of beige adipocytes with a gene expression pattern distinct from either white or brown adipocytes has been described. Adipocytes can synthesize estrogens from androgens, potentially being the reason why being underweight or overweight are risk factors for infertility. Additionally, adipocytes are responsible for the production of the hormone leptin, an important in regulation of appetite and acts as a satiety factor.

Adipocytes may be identified by expression of specific markers including 4-1BB/TNFRSF9/CD137, Adiponectin/Acrp30, gAdiponectin/gAcrp30, AdipoR1, AdipoR2, CIDEA, Clathrin Heavy Chain 2/CHC22, DLK2/EGFL9, FABP4/A-FABP, FATP1, FATP2, FATP4, FATP5, FATP6, Glut4, Leptin/OB, Perilipin-2, PGC1 alpha, PPAR gamma/NR1C3, Pref-1/DLK1/FA1, Seipin/BSCL2, UCP1, and/or ZIC1.

Chondrocytes are found in healthy cartilage. The cells produce and maintain the cartilaginous matrix, which consists mainly of collagen and proteoglycans. Although the word chondroblast is commonly used to describe an immature chondrocyte, the term is imprecise, since the progenitor of chondrocytes (which are mesenchymal stem cells) can differentiate into various cell types, including osteoblasts.

Chondrocytes may be identified by expression of specific markers including Aggrecan, Annexin A6, Cathepsin B, CD44, CD151, Chondroadherin, Collagen II, Collagen IV, Collagen IV alpha 1, CRTAC1, DSPG3, FAM20B, FoxC1, FoxC2, IBSP/Sialoprotein II, ITM2A, Matrilin-1, Matrilin-3, Matrilin-4, MIA, Otoraplin/OTOR, SOX5, SOX6, SOX9, and/or URB.

Melanocytes are melanin-producing cells located in the bottom layer (the stratum basale) of the skin's epidermis, the middle layer of the eye (the uvea), the inner ear, meninges, bones, and heart. Melanin is the pigment primarily responsible for skin color. Once synthesized, melanin is contained in a special organelle called a melanosome and moved along arm-like structures called dendrites, so as to reach the keratinocytes.

Schwann cells are the principal glia of the peripheral nervous system (PNS). Glial cells function to support neurons and in the PNS, also include satellite cells, olfactory ensheathing cells, enteric glia and glia that reside at sensory nerve endings, such as the Pacinian corpuscle. There are two types of Schwann cell, myelinating and nonmyelinating. Myelinating Schwann cells wrap around axons of motor and sensory neurons to form the myelin sheath. The Schwann cell promoter is present in the downstream region of the Human Dystrophin Gene that gives shortened transcript that are again synthesized in a tissue specific manner. Schwann cells are involved in many important aspects of peripheral nerve biology—the conduction of nervous impulses along axons, nerve development and regeneration, trophic support for neurons, production of the nerve extracellular matrix, modulation of neuromuscular synaptic activity, and presentation of antigens to T-lymphocytes. Charcot-Marie-Tooth disease (CMT), Guillain-Barré syndrome (GBS, acute inflammatory demyelinating polyradiculopathy type), schwannomatosis, and chronic inflammatory demyelinating polyneuropathy (CIDP), and leprosy are all neuropathies involving Schwann cells.

Schwann cells may be identified by expression of specific markers including alpha 2a Adrenergic Receptor, Calretinin, ChAT, MAG/Siglec 4a, Neurofilament NF-H, Neurofilament NF-L, Neurofilament NF-M and/or Neurofilament alpha-intemexin/NF66.

In one embodiment, the present invention provides for a method of differentiating human pluripotent stem cells (hPSCs) into neural crest stem cells (NCSCs) comprising culturing hPSCs with at least two agents including a rho-associated protein kinase (ROCK) inhibitor, a glycogen synthase kinase 3 (GSK-3) inhibitor, an activing receptor-like kinase (ALK) receptor inhibitor and/or a bone morphogenic protein (BMP) receptor inhibitor, under conditions for such time as to allow the agents to effect differentiation of the hPSCs. In one aspect, the hPSCs are parthenogenetic stem cells (hpSCs), induced pluripotent stem cells (iPSCs), nuclear transfer stem cells, adult stem cells or embryonic stem cells. In another aspect, the ALK inhibitor inhibits ALK4, ALK5 and/or ALK7 and the BMP receptor inhibitor inhibits ALK2. In an additional aspect, the ROCK inhibitor is Y27632, AS1 892802, GSK 269962, GSK 429286, H 1152, HA 1100 hydrochloride, OXA 06 dihydrochloride, RKI 1447 dihydrocholoride, SB 772077B dihydrocholoride, SR 3677 dihdrochloride, or TC-S 7001, the GSK-3 inhibitor is Chir99021, 3F8, A 1070722, AR-A 014418, BIO, BIO-acetoxime, 10Z-Hymenialdisine, Indirubin-3′-oxime, Kenpaullone, Lithium carbonate, NSC 693868, SB216763, SB 415286, TC-G 24, TCS 2002, TCS21311, or TWS 119, the ALK inhibitor is SB43152, A 83-01, D 4476, GW 788388, LY 364974, R 268712, RepSox, SB 505124, SB 525334, or SD 208 and the BMP receptor inhibitor is DMH-1, DMH2, Dorsomorphin dihydrochloride, K 02288, or ML 347. In a further aspect, the hPSCs are contacted with at least three agents. In a specific aspect, the at least three agents are Y27632, Chir99021, SB43152 and/or DMH-1. In an additional aspect, the hPSCs are contacted with at least four agents. In a specific aspect, the at least four agents are Y27632, Chir99021, SB43152 and DMH-1. In a further aspect, the NCSCs express at least one neural crest cell marker and at least one marker of pluripotency. In one aspect, the at least one neural crest cell marker of differentiation is PAX3, P75, NGFR, SOX10, FOXD3, NESTIN, SNAI2, Ki67 or HNK-1 and the at least one marker of pluripotency is NANOG, ZNF206, or OCT4. In an additional aspect, the hPSCs are contacted with the at least two agents for at least about 1, 2, 3, 4, 5, 6, 7, 8, 9, or 10 days. In a preferred aspect, the contacting is for at least about 6 days. In another aspect, the NCSCs are capable of being maintained in an undifferentiated state for at least about 1, 2, 3, 4, 5, 6, 7, 8, 9, 10, 15, 20 or 25 passages. In a specific aspect, the NCSCs are capable of being maintained in an undifferentiated state for at least about 5 passages. In an additional aspect, the NCSCs are differentiated into astrocytes, smooth muscle cells, osteoblast, adipocytes, chondrocytes, melanocytes, Schwann cells and/or neurons. In certain aspects, the astrocytes express S100β, HNK1 and/or GFAP; the smooth muscle cells express Caldesmon, P75 and/or SMA and the neurons express MAP2, SOX10 and/or TUJ1. In one aspect, the hPSCs are cultured in a media comprising StemLife MSC basal medium, Glutamax, B27, Y27632, CHIR99021, SB43152 and DMH-1.

In another embodiment, the present invention provides a method of treating neurocristopathic disease or disorder comprising obtaining human pluripotent stem cells (hPSCs); contacting the hPSCs with at least two agents selected from the group consisting of a rho-associated protein kinase (ROCK) inhibitor, a glycogen synthase kinase 3 (GSK-3) inhibitor, an activing receptor-like kinase (ALK) receptor inhibitor and/or a bone morphogenic protein (BMP) receptor inhibitor to differentiate the hPSCs into neural crest stem cells (NCSCs) under conditions and for such time as to allow the agents to effect differentiation of the hPSCs; and administering the NCSCs to a subject in need thereof. In one aspect, the neurocristopathic disease or disorder is piebaldism, Waardenburg syndrome, Hirschsprung disease, Ondine's curse (congenital central hypoventilation syndrome), pheochromocytoma, paraganglioma, Merkel cell carcinoma, multiple endocrine neoplasia, neurofibromatosis type I, CHARGE syndrome, familial dysautonomia, DiGeorge syndrome, Axenfeld-Rieger syndrome, Goldenhar syndrome (a.k.a. hemifacial microsomia), craniofrontonasal syndrome, congenital melanocytic nevus, melanoma, or congenital heart defects of the outflow track. In another aspect, the neurocristopathic disease or disorder is Waardenburg syndrome or Hirschsprung disease. In an additional aspect, the hPSCs are parthenogenetic stem cells (hpSCs), induced pluripotent stem cells (iPSCs), nuclear transfer stern cells, adult stem cells or embryonic stem cells. In a further aspect, the ROCK inhibitor is Y27632, the GSK-3 inhibitor is Chir99021, ALK receptor inhibitor is SB43152 and the BMP receptor inhibitor is DMH-1. In one aspect, the NCSCs express at least one neural crest cell marker wherein the neural crest stem cell marker is PAX3, P75 NGFR, SOX10, FOXD3, NESTIN, SNAI2, Ki67 or HNK-1 and the at least one marker of pluripotency is NANOG, ZNF206, or OCT4. In another aspect, the contacting is for at least about 6 days and the NCSCs are capable of being maintained in an undifferentiated state for at least about 5 passages. In a further aspect, the NCSCs are differentiated into astrocytes, smooth muscle cells, osteoblast, adipocytes, chondrocytes, melanocytes, Schwann cells and/or neurons.

In an additional embodiment, the invention provides for a kit for the differentiation of human pluripotent stem cells (hPSCs) into neural crest stem cells (NCSCs) comprising of a rho-associated protein kinase (ROCK) inhibitor, a glycogen synthase kinase 3 (GSK-3) inhibitor, an activing receptor-like kinase (ALK) receptor inhibitor and a bone morphogenic protein (BMP) receptor inhibitor and instructions. In one aspect, ROCK inhibitor is Y27632, the GSK-3 inhibitor is Chir99021, the ALK receptor inhibitor is SB43152 and the BMP receptor inhibitor is DMH-1.

As described in the Examples, the disclosed methods generate multipotent NCSCs from human parthenogenetic stem cells (hpSCs). The derived NCSCs express markers specific for NCSCs as well as markers of pluripotency. Further, the derived NCSCs were further differentiated into astrocytes, neurons and smooth muscle cells.

The following examples are intended to illustrate, but not limit the invention.

### EXAMPLE 1

Feeder Growth of Human Parthogenetic Stem Cells (hpSCs). The hpSCs were first maintained on mitomycin-C inactivated mouse embryonic fibroblast (Millipore) feeder layer in embryonic stem medium: Knock Out DMEM/F12 (Life Technologies), 2 mM L-glutamine (GlutaMax-I, Invitrogen), 0.1 mM MEM nonessential amino acids (Life Technology), 0.1 mM β-mercaptoethanol (Life Technologies), penicillin/streptomycin/amphotericin B (100 U/100 μg/250 ng) (MP Biomedicals) and 5 ng/ml bFGF (Peprotech). Cells were passaged with dispase or collagenase IV (both Life Technologies) every 5-7 days with split ratio of 1:4 or 1:6

Feeder-Free Growth of hpSCs. The hpSC line LLC2PH were then transferred to Vitronectin (BD Biosciences) coated plates and grown with Essential 8 medium (Invitrogen).

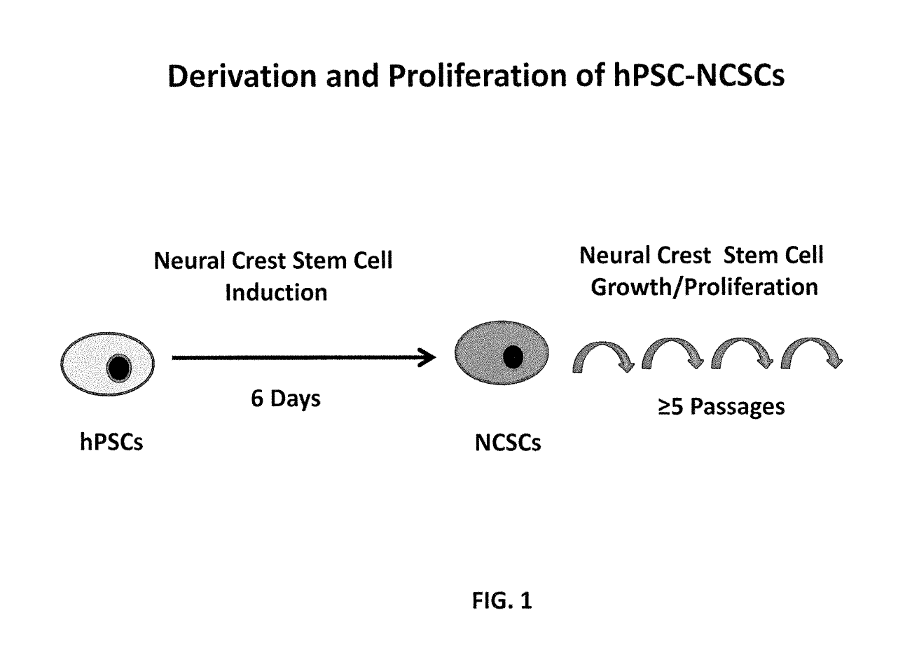

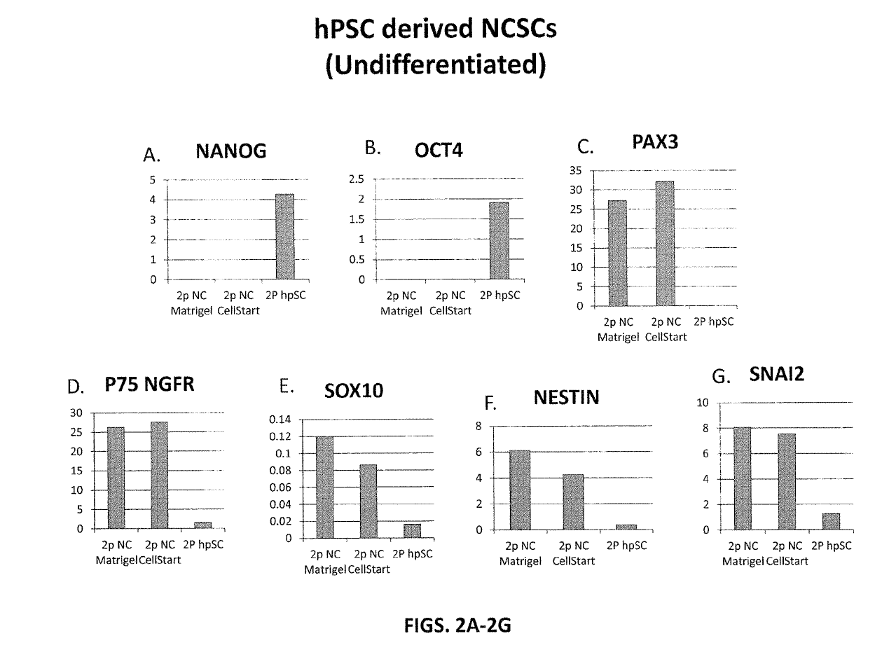

Neural Crest Stern Cell (NCSC) Derivation and Growth. Neural crest induction is performed by treating proliferating, 80-90% confluent feeder-free hpSCs cultures, with DMEM/F12-GlutaMAX basal medium supplemented with 1X B27 supplement, 1X N2 supplement plus a chemical cocktail consisting of four small molecules Y27632 (10 μM), CHIR99021 (5 μM), SB43152 (5 μM) and DMH-1 (2 μM) for 6 days (FIG. 1). After 6 days, hpSC-neural crest induced cells are treated with Accutase and then grown on either Matrigel or CELLstart coated plates in StemLife MSC basal medium supplemented with 1× Glutamax, 1× B27 supplement plus a chemical cocktail consisting of four small molecules Y27632 (10 μM), CHIR99021 (5 μM), SB43152 (5 μM) and DMH-1 (2 μM) (FIG. 1). RT-PCR gene expression analysis of human parthenogenetic derived neural crest stem cells was performed for genes associated with the neural crest cell lineage SOX10, FINK-1, P75, PAX3, SNAI2, Nestin and the pluripotency genes NANOG and OCT4. Immunoflourescence images of human parthenogenetic derived neural crest stem cells fixed and stained for proteins associated with the neural crest stem cell lineage: SOX10, HNK-1, P75, NESTIN and Ki67. The hpSCs-NCSCs lose expression of pluripotency genes NANOG and OCT4 after 6 days and upregulate the expression of neural crest stem cell associated genes SOX10, HNK-1, P75, PAX3, SNAI2, Nestin and Ki67 (FIG. 2).

hPSC Derived NCSCs are Multipotent. To determine whether hPSC derived NCSCs are multipotent, the cells were plated on Matrigel coated plates and were differentiated with a medium consisting of StemLife MSC basal medium supplemented with 1× Gultamax and 1× B27 Supplement (without chemicals). After two weeks, the cells were fixed and stained for astrocyte (GFAP), smooth muscle cell (Smooth Muscle Actin) and neuronal (TUJ1) cell lineage markers. Immunoflourescence images showed the differentiation of hpNCSCs into Glial (GFAP), smooth muscle (SMA) and neuronal differentiation (TUJ1) cell lineages.

Although the invention has been described with reference to the above example, it will be understood that modifications and variations are encompassed within the spirit and scope of the invention. Accordingly, the invention is limited only by the following claims.

What is claimed is:

1. A method of differentiating human pluripotent stem cells (hPSCs) into neural crest stem cells (NCSCs) comprising culturing hPSCs with at least two agents selected from the group consisting of a rho-associated protein kinase inhibitor (ROCK), a glycogen synthase kinase 3 (GSK-3) inhibitor, an activing receptor-like kinase (ALK) receptor inhibitor and a bone morphogenic protein (BMP) receptor inhibitor, under conditions for such time as to allow the agents to effect differentiation of the hPSCs.

2. The method of claim 1, wherein the hPSCs are selected from the group consisting of parthenogenetic stem cells (hpSCs), induced pluripotent stem cells (iPSCs), nuclear transfer stem cells, adult stem cells and embryonic stem cells (hES).

3. The method of claim 1, wherein the ALK inhibitor inhibits ALK4, ALK5 and/or ALK7 and the BMP receptor inhibitor inhibits ALK2.

4. The method of claim 1, wherein the ROCK inhibitor is selected from the group consisting of Y27632, AS1 892802, GSK 269962, GSK 429286, H 1152, HA 1100 hydrochloride, OXA 06 dihydrochloride, RKI 1447 dihydrocholoride, SB 772077B dihydrocholoride, SR 3677 dihdrochloride, and TC-S 7001, the GSK-3 inhibitor is selected from the group consisting of Chir99021, 3F8, A 1070722, AR-A 014418, BIO, BIO-acetoxime, 10Z-Hymenialdisine, Indirubin-3′-oxime, Kenpaullone, Lithium carbonate,NSC 693868, SB216763, SB 415286, TC-G 24, TCS 2002, TCS21311, and TWS 119, the ALK inhibitor is selected from the group consisting of SB43152, A 83-01, D 4476, GW 788388, LY 364974, R 268712, RepSox, SB 505124, SB 525334, and SD 208 and the BMP receptor inhibitor is selected from the group consisting of DMH-1, DMH2, Dorsomorphin dihydrochloride, K 02288, and ML 347.

5. The method of claim 1, wherein the hPSCs are contacted with at least three agents.

6. The method of claim 5, wherein the at least three small molecule compounds are selected from the group consisting of Y27632, Chir99021, SB43152 and DMH-1.

7. The method of claim 1, wherein the NCSCs express at least one neural crest cell marker and at least one marker of pluripotency.

8. The method of claim 6, wherein the at least one neural crest cell marker of differentiation is selected from the group consisting of PAX3, P75, NGFR, SOX10, FOXD3, NESTIN, SNAI2, Ki67 and FINK-1 and wherein the at least one marker of pluripotency is selected from the group consisting of NANOG, ZNF206, and OCT4.

9. The method of claim 1, wherein the hPSCs are contacted with the at least two agents for at least about 1, 2, 3, 4, 5, 6, 7, 8, 9, or 10 days.

10. The method of claim 9, wherein the hPSCs are contacted with the at least two agents for at least about 6 days.

11. The method of claim 1, wherein the NCSCs are capable of being maintained in an undifferentiated state for at least about 1, 2, 3, 4, 5, 6, 7, 8, 9, 10, 15, 20 or 25 passages.

12. The method of claim 11, wherein the NCSCs are capable of being maintained in an undifferentiated state for at least about 5 passages.

13. The method of claim 1, further comprising differentiating the NCSCs into astrocytes, smooth muscle cells, osteoblast, adipocytes, chondrocytes, melanocytes, Schwann cells and/or neurons.

14. The method of claim 13, wherein the astrocytes express S100β, HNK1 and/or GFAP; the smooth muscle cells express Caldesmon, P75 and/or SMA and the neurons express Map2, SOX10 and/or TUJ1.

15. A method of treating neurocristopathic disease or disorder comprising:
a) obtaining human pluripotent stem cells (hPSCs);
b) culturing the hPSCs with at least two agents selected from the group consisting of a rho-associated protein kinase (ROCK) inhibitor, a glycogen synthase 3 (GSK-3) inhibitor, an activing receptor-like kinase (ALK) receptor inhibitor and a bone morphogenic protein (BMP) receptor inhibitor to differentiate the hPSCs into neural crest stem cells (NCSCs) under conditions for such time as to allow the agents to effect differentiation of the hPSCs; and
c) administering the NCSCs to a subject in need thereof.

16. The method of claim 15, wherein the neurocristopathic disease or disorder is selected from the group consisting of piebaldism, Waardenburg syndrome, Hirschsprung disease, Ondine's curse (congenital central hypoventilation syndrome), pheochromocytoma, paraganglioma, Merkel cell carcinoma, multiple endocrine neoplasia, neurofibromatosis type I, CHARGE syndrome, familial dysautonomia, DiGeorge syndrome, Axenfeld-Rieger syndrome, Goldenhar syndrome (a.k.a. hemifacial microsomia), craniofrontonasal syndrome, congenital melanocytic nevus, melanoma, and congenital heart defects of the outflow track.

17. The method of claim 16, wherein the neurocristopathic disease or disorder is Waardenburg syndrome or Hirschsprung disease.

18. The method of claim 15, wherein the hPSCs are selected from the group consisting of parthenogenetic stem cells (hpSCs), induced pluripotent stem cells (iPSCs), nuclear transfer stem cells, adult stem cells and embryonic stem cells (hES).

19. The method of claim 15, wherein the ROCK inhibitor is Y27632, the GSK-3 inhibitor is Chir99021, the ALK inhibitor is SB43152, and the BMP receptor inhibitor is DMH-1

20. The method of claim 15, wherein the NCSCs express at least one neural crest cell marker selected from the group consisting of PAX3, P75 NGFR, SOX10, FOXD3, NESTIN, SNAI2, Ki67 and HNK-1 and at least one marker of pluripotency selected from the group consisting of NANOG, ZNF206 and OCT4.

21. The method of claim 15, wherein the hPSCs are contacted with the at least two agents for at least about 6 days.

22. The method of claim 15, wherein the NCSCs are capable of being maintained in an undifferentiated state for at least about 5 passages.

23. The method of claim 15, further comprising differentiating the NCSCs into astrocytes, smooth muscle cells, osteoblast, adipocytes, chondrocytes, melanocytes, Schwann cells and/or neurons.

24. A kit for the differentiation of human pluripotent stem cells (hPSCs) into neural crest stem cells (NCSCs) comprising of a rho-associated protein kinase inhibitor, a GSK-3 inhibitor, an activing receptor-like kinase (ALK) receptor inhibitor and a bone morphogenic protein (BMP) receptor inhibitor and instructions.

25. The kit of claim 24, wherein the ROCK inhibitor is Y27632, the GSK-3 inhibitor is Chir99021, the ALK receptor inhibitor is SB43152 and the BMP receptor inhibitor is DMH-1.
